(19) United States
(12) Patent Application Publication
(10) Pub. No.: US 2015/0164558 A1
(43) Pub. Date: Jun. 18, 2015

(54) BONE ANCHOR CLOSURE PIVOT-SPLAY EFFECT SHIFTING GUIDE AND ADVANCEMENT STRUCTURE WITH MODIFIED SQUARE THREAD

(71) Applicants: Roger P. Jackson, Prairie Village, KS (US); James L. Surber, Kansas City, KS (US)

(72) Inventors: Roger P. Jackson, Prairie Village, KS (US); James L. Surber, Kansas City, KS (US)

(21) Appl. No.: 14/569,142

(22) Filed: Dec. 12, 2014

Related U.S. Application Data
(60) Provisional application No. 61/915,085, filed on Dec. 12, 2013.

(51) Int. Cl. A61B 17/70

(52) U.S. Cl. CPC A61B 17/7037; A61B 17/7032

(57) ABSTRACT

An open implant closure mechanism includes a closure top with a square thread form in combination with a mating substantially similar thread form on a bone anchor having a load flank disposed at a reverse angle.

### CROSS-REFERENCE TO RELATED APPLICATIONS

This application claims the benefit of U.S. Provisional Patent Application Ser. No. 61/915,085, filed Dec. 12, 2013, which is incorporated by reference herein.

### BACKGROUND OF THE INVENTION

The present invention is directed to structure for joining together parts of a medical implant, in particular for use with open bone anchors in spinal surgery, and in some embodiments thereof, for use with spinal bone anchors such as polyaxial screws with U-shaped rod-accepting receivers having upwardly extending arms with helically wound guide and advancement structures.

Bone anchors, such as bone screws and hooks are utilized in many types of spinal surgery in order to secure various implants to vertebrae along the spinal column for the purpose of stabilizing and/or adjusting spinal alignment. For example, the most common mechanism for providing vertebral support is to implant bone screws into certain bones which then in turn support a longitudinal connecting member, such as a rod connector, or are supported by the connector. Although both closed-ended and open-ended bone anchors are known, open-ended anchors are particularly well suited for connections to longitudinal connecting members such as hard, soft or deformable rods, dynamic, soft or elastic connectors and connector sleeves or arms, because such rods or other connector members do not need to be passed through a closed bore, but rather can be laid or urged into an open channel within a U-shaped receiver or head of such a bone anchor. Generally, the anchors must be inserted into the bone as an integral unit or a preassembled unit, in the form of a shank or hook and connected pivotal receiver. In some instances, a portion of such a preassembled unit, such as a shank of a polyaxial bone screw assembly, may be independently implanted into bone, followed by push- or pop-on assembly of a receiver portion of the unit that includes the open channel for receiving a rod connector or other longitudinal connecting member.

Typical open-ended bone screws include a threaded shank with a head or receiver having a pair of parallel projecting branches or arms which form a yoke with a U-shaped slot or channel to receive a portion of a rod or other longitudinal connecting member. Hooks and other types of connectors, as are used in spinal fixation techniques, may also include similar open ends for receiving rods or portions of other fixation and stabilization structure. After the rod or other longitudinal connecting member is placed in the receiver channel, a closure, typically in the form of a substantially cylindrical plug is often used to close the channel. Known closures include slide-on types, twist-on varieties that are rotated ninety degrees to a locked in position, and a variety of single start helically wound guide and advancement structures including, for example, thread forms having v-thread, reverse-angle, buttress or square thread forms, to name a few, as well as other non-threadlike helically wound forms, such as helical flanges.

It is known that the angled loading flank of a v-thread closure generates outward splay of spaced open implant receiver arms at all loading levels without limit. Thus, v-threaded closures or plugs are sometimes used in combination with outer threaded nuts that prevent outward splaying of the receiver arms. To overcome the splay problems of v-threaded closures, so-called “buttress” thread forms were developed. In a buttress thread, the trailing or thrust surface of the closure is linear and oriented somewhat downwardly in the direction of advancement with respect to the thread axis, while the leading or clearance surface is angled rearwardly in varying degrees, theoretically resulting in a neutral radial reaction of a threaded receptacle or receiver to torque on the threaded closure member being received thereby. In reverse angled thread forms, which theoretically positively draw the threads of a receptacle radially inwardly toward the thread axis when the reverse angle closure thread is torqued, provided the outer tip of the thread is crested and strong enough, the trailing linear surface of the external thread of the closure is angled toward the thread axis instead of away from the thread axis (as in conventional v-threads). Although buttress and reverse angle threads with linear loading surfaces reduce the tendency of bone screw receiver arms to splay outwardly, they are not structured to control it and the arms may still be flexed outwardly by bending moment forces acting on the implant arms and the closure threads can be bent, deformed or even sheared off by forces exerted during installation, as well as experienced post-operatively on the implants with certain activities.

Closures made with square threads, again, having linear loading surfaces, theoretically keep all forces axially directed. However, it has been found that under a moderate load, square thread closures produce a marginal splay and under heavy load, splay can be considerable, indicating that traditional square thread machine design theories directed to power screws and other screws for use in substantially closed bores do not adequately describe and reflect the environment of a spinal open bone screw receiver having a relatively small size with spaced apart arms in lieu of a bore. Furthermore, square threaded spinal bone anchor closures have been shown to experience heretofore unexplained and undesirable excess stress concentrated on their upper outer loading flank portions located near the crest of the thread, believed by Applicant to be due to outwardly directed rotational pivot and displacement of the mating receiver arm thread flank portions when under the stress of loading between the arms of an open receiver. This occurs during intra-operative tightening and post-operative physiologic loading, which is believed to have resulted in in-vivo loosening in some cases.

### SUMMARY OF THE INVENTION

A mechanism according to an embodiment of the invention for capturing and fixing a longitudinal connecting member, such as a rod, within a bone anchor having spaced arms includes an open receiver with a first helically wound thread form and a closure having a helically wound substantially horizontal square thread form. The receiver guide and advancement structure is a modified square-like thread form that does not mirror the angular orientation of the closure square thread form, the receiver thread form having a loading flank oriented at a slight reverse angle and an evenly spaced opposed and substantially parallel stab flank. The orientation of the receiver thread loading flank is configured to accommodate a thread pivot and tilt that occurs during outward rotational splay of the receiver arms. With such modification of the receiver thread form, upward and downward facing receiver thread flanks remain parallel to one another; however, the opposed closure and receiver loading flanks are initially in a non-parallel contact relationship with contact between such flanks located more medially on the closure thread than possible with a traditional square thread receiver.

More specifically, according to an aspect of the invention, the receiver thread form includes a discontinuous receiver guide and advancement thread form extending helically about and along an inner surface of each receiver arm, the receiver thread form having a slightly reverse angle load flank engaging a substantially horizontal load flank of the closure during mating of the closure square thread form with the receiver thread form. The receiver thread form upward and downward facing flanks are parallel to one another. However, the opposed closure and receiver loading flanks are initially in a non-parallel contact relationship early in the loading process with contact between the opposed loading surfaces located near a root of the closure thread form and thus more favorably on the closure thread, delaying contact near the crest of the closure thread form until late in the loading cycle, at which point the loading flanks are flush following the predetermined pivot-splay that occurs in the receiver arms.

Objects of the invention further include providing apparatus and methods that are easy to use and especially adapted for the intended use thereof and wherein the tools are comparatively inexpensive to produce. Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is front elevational view of a portion of a spinal implant showing only a rod receiver and a closure and with portions broken away to show the detail of a prior art helical square thread mechanism located on the closure and the receiver.

FIG. 2 is an enlarged and partial front elevational view with portions broken away of the prior art square thread closure mechanism of FIG. 1 illustrating outward pivot-splay of the receiver.

FIG. 3 is a further enlarged and partial view with portions broken away of the prior art mechanism of FIG. 2.

FIG. 4 is a further enlarged and partial view of the prior art mechanism of FIG. 3 further illustrating corner or crest loading of the closure square thread.

FIG. 5 is a front elevational view of a portion of a spinal implant showing only a rod receiver and a closure with portions broken away to show the detail of an embodiment of a mating square thread mechanism according to an aspect of the invention and shown with the closure in mating but unloaded engagement with the receiver.

FIG. 6 is an enlarged and partial front elevational view with portions broken away of the mechanism of FIG. 5.

FIG. 7 is another enlarged and partial front elevational view with portions broken away of the mechanism of FIG. 5 shown in a loaded relationship.

### DETAILED DESCRIPTION OF THE INVENTION

As required, detailed embodiments are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the bone attachment structures in actual use.

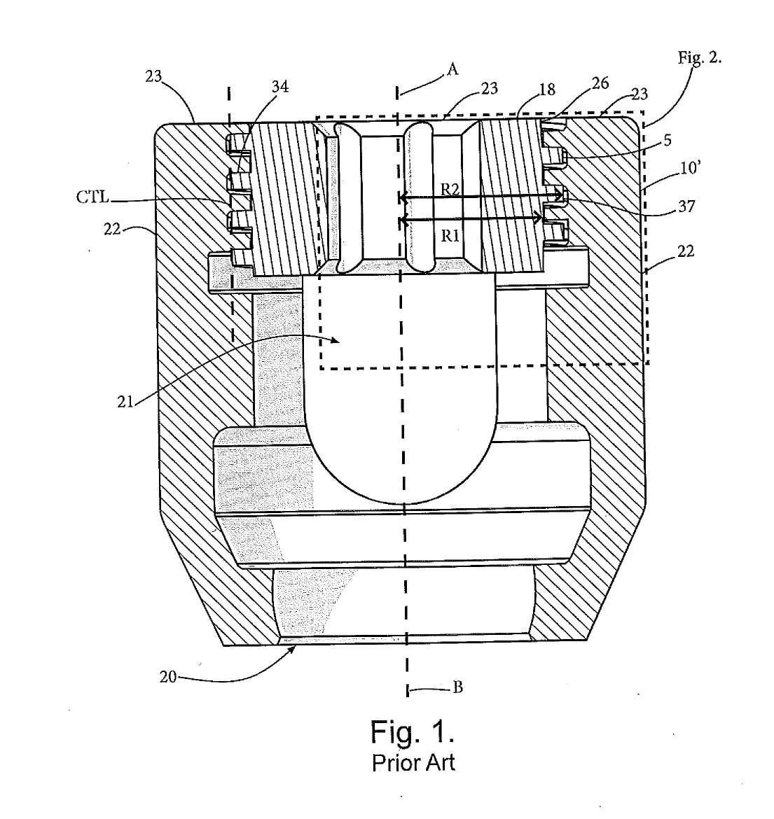

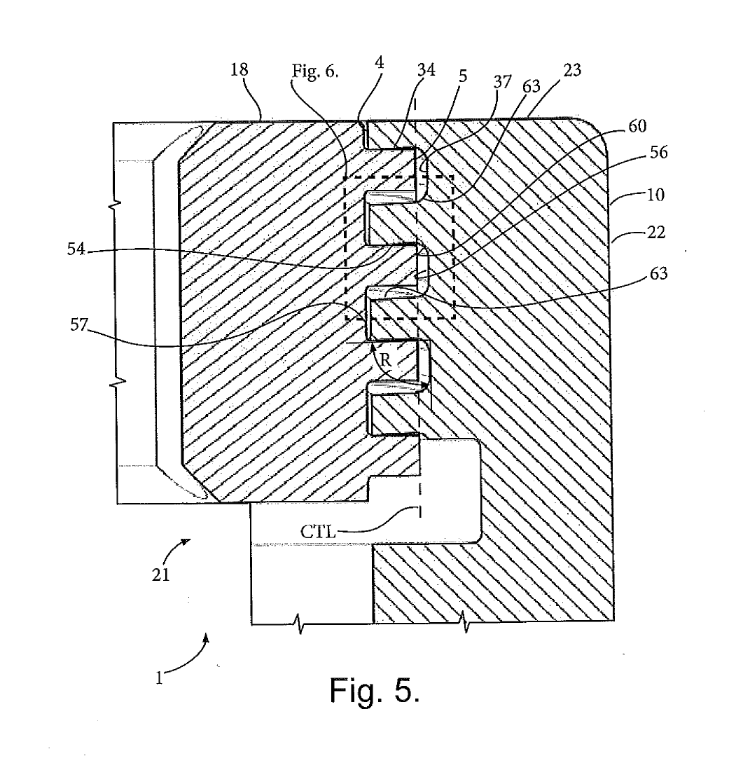

The reference numeral 1 generally indicates a closure mechanism in accordance with an embodiment of the invention that includes a first or female helical thread form, generally 4, mated with a second or male square thread form, generally 5. The thread form 4 is located on a receiver 10 illustrated in FIGS. 5-7 of an open bone anchor (not otherwise shown) and the square thread form 5 is located on a closure 18 that is shown in all of the FIGS. 1-7. The closure 18 illustrated in this application is shown with a prior art open implant receiver 10′ in FIGS. 1-4 and with an embodiment of a receiver 10 that includes the thread form 4 according to an embodiment of the invention in FIGS. 5-7. The illustrated receiver 10 is identical to the illustrated prior art receiver 10′ with the exception of the thread form 4. Thus, in all of the drawing figures described herein, various structure and aspects of the receiver 10 and the receiver 10′ shall be identified with the same reference numbers with the exception of the prior art square thread form on the receive 10′ that is identified as 4′ (and “′” will be used to identify all detailed features of the thread form 4′).

The receiver 10 (and receiver 10′) is further part of a polyaxial bone screw apparatus or assembly that includes a shank (not shown) with a body having a bone engaging and implantation thread and also an upwardly extending substantially or partially spherical upper portion or head. The receiver 10 has a cavity or inner chamber, generally 20, for receiving such a shank head, the cavity 20 communicating with an upper channel 21 formed between opposed arms 22 having top surfaces 25. The discontinuous thread form 4 is located near the top surface 23 of each arm 22 and faces inwardly toward the channel 21, winding helically downwardly in a direction toward the cavity 20 and thus defining an upper portion of the channel 21. Similarly, the prior art thread form 4′ is located near the top surface of the receiver 10′ and defines a portion of the channel 21 of the receiver 10′. A bone screw assembly having the closure mechanism 1 may further include a compression or pressure insert (not shown) having a lower surface engaging the shank head within the receiver cavity. Such an insert may also define an inner channel between opposed arms for receiving a cylindrical surface of a rod, for example. For example, an assembly as shown in Applicant's U.S. patent application Ser. No. 13/317,969 filed Nov. 1, 2011, and incorporated by reference herein, may be modified for use with the closure mechanism 1 of the invention.

It is noted that closures for use with the closure mechanism 1 may take a variety of forms, including single and multi-start options, one piece closures, two piece closures, closures with break-off heads, for example, and may be used with a wide variety of medical implants, including, but not limited to mono-axial screws and hooks, hinged or uni-planar screws and hooks and dual multi-piece polyaxial bone screws and hooks, as well as screws with sliding or pivoting inserts. A variety of polyaxial bone screws may also be used with closure mechanisms of the invention and the illustrated embodiment should not be considered limiting. For example, mechanisms or structures 1 of the invention may be used with bone screws having top loaded bone screw shanks with spherical heads and also with bottom-loaded multi-part screw shanks as well as other types of bottom loaded screws including screws that may be initially implanted into bone followed by press-on or snap-on of a remainder of the bone anchor that may include an open retaining ring or some form of collet structure for capturing the bone screw head within the receiver.

The illustrated square thread closure 18 that is sectionally shown in all of the FIGS. 1-7 is in the form of a cylindrical plug, with the square thread 5 winding helically along an outer surface 26 thereof and about an axis of rotation A of the closure 18. As will be described in greater detail below, the outer structure 5 of the closure top 18 mates under rotation with the receiver 10 having a central axis B with the axis A being aligned with the axis B, the closure top 18 pressing downwardly upon a rod or other longitudinal connecting member that in turn presses upon the shank head (either directly or indirectly via an insert), placing the head into a fixed position with respect to the receiver 10, locking the polyaxial mechanism of the bone anchor, (i.e., fixing the shank at a particular angle with respect to the receiver 10). The closure 18 ultimately frictionally engages and presses against the longitudinal connecting member, for example, a rod, so as to capture, and fix the longitudinal connecting member within the receiver 10 and thus fix the member relative to a vertebra (not shown).

The rod or other longitudinal connecting member may be hard, stiff, non-elastic and is typically cylindrical. However, a longitudinal connecting member for use with the assembly may take the form of an elastic or deformable cylinder or have a different cross-sectional geometry. The longitudinal connecting member may also be a part of a soft or dynamic system that may include hard or soft structure for attaching to the assembly and may further include a tensioned cord, elastic bumpers and spacers located between bone screws, for example. The illustrated receiver 10 and the shank (not shown) may cooperate in such a manner that the receiver 10 and the shank can be secured at any of a plurality of angles, articulations or rotational alignments relative to one another and within a selected range of angles both from side to side and from front to rear, to enable flexible or articulated engagement of the receiver 10 with the shank until both are locked or fixed relative to each other near the end of an implantation procedure.

Returning to FIGS. 1-7, the square thread closure 18 helical thread 5 is illustrated as a single start structure that includes several surfaces that helically wrap about the axis A. The surface 26 upon which the form 5 helically winds may also be defined as a root surface that is helical and disposed substantially parallel to the axis A. A virtual cylinder formed by the root surface 26 has a radius R1 (radial distance between the axis A and the surface 26). Adjacent the root surface 26 is a radiused surface, curve or corner surface 30 that in turn is adjacent to a load or loading surface or helical flank 34. The load flank 34 extends substantially perpendicular to the axis A and thus runs substantially perpendicular about the root surface 26 and is on a trailing side relative to a direction of advancement of the structure 5 along the receiver axis B when the structure 5 rotatingly mates with the receiver thread form 4 on the receiver arms 22. The load flank 34 extends outwardly to a crest surface 37, the surfaces 34 and 37 intersecting at an outer corner or edge 40. A substantial portion of the crest surface 37 is substantially parallel to the root surface 26. Thus, a virtual cylinder formed by the crest surface 37 has a radius R2 (radial distance between the axis A and the surface 37).

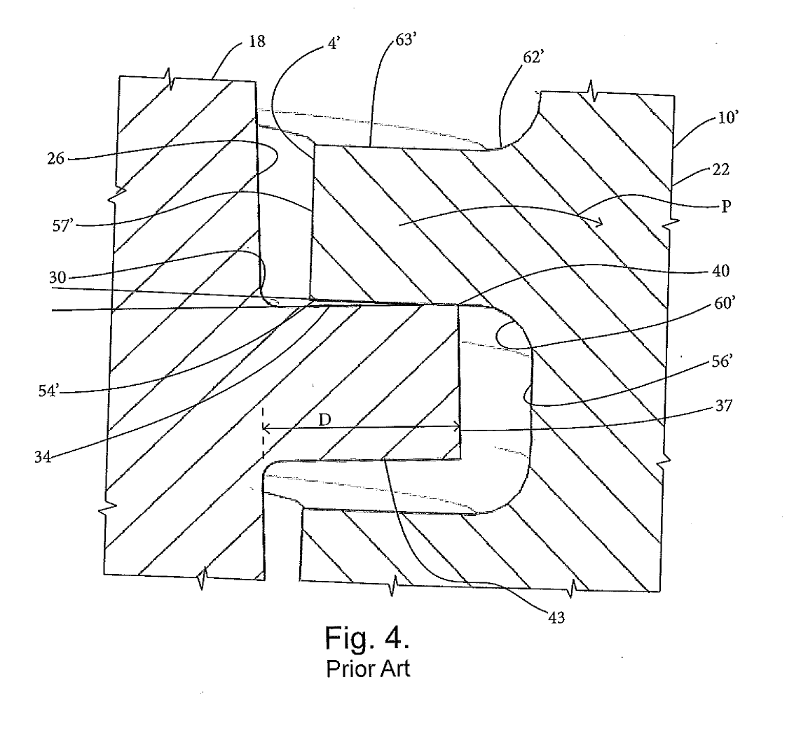

With further reference to FIG. 4, a distance D identifies a depth of the thread form 5 from the crest 37 to the root 26. Stated in another way, D=R2−R1. It is noted that by combining beam and thread theory it is believed that a larger core diameter for the closure 18 and a shorter thread depth D thereof is advantageous in decreasing bending moments on the receiver arms 22. This is because such thread loading flanks are positioned closer to a central tension line CTL (see, e.g., FIG. 1 that shows a theoretical central tension line for the receiver 10′ and FIG. 5 for the receiver 10) within the upwardly extending arms 22 of the U-shaped receiver 10. Moving the root of the male closure thread outwardly as close as possible to the theoretical tension line CTL in the receiver 10 arms is desirable in that it can decrease the resultant bending moment on the receiver arms 22. By so doing, the outer diameter of the closure top (measured between crest surfaces 37 can remain the same size for a given receiver 10.

A lower stab surface or flank 43 is uniformly spaced from and runs parallel to the loading flank 34 and extends from the root surface 26 to the crest surface 37. The surface 43 is perpendicular to the axis A and thus perpendicular to the surfaces 26 and 37. The stab flank 43 is located opposite the load flank 34. The load flank 34 may also be referred to as a thrust surface while the stab flank 43 may also be referred to as a clearance surface. To complete the illustrated square thread 5 geometry, a curved inner corner surface 46 joins the stab surface 43 to the root surface 26.

With particular reference to FIGS. 5-7, the thread form 4 located on each receiver arm 22 cooperates with the form 5, but, unlike the prior art square thread form 4′ shown on the receiver 10′ in FIGS. 1-4, the form 4 is not identical to the form 5 nor even a mirror image thereof. Rather, to create the form 4, the square thread form 4′ of the receiver 10′ is modified at a loading flank thereof as will be described in greater detail below to counter a pivot-splay force that is believed to cause stress and deformation on the male thread 5 of the closure as will also be described in greater detail below.

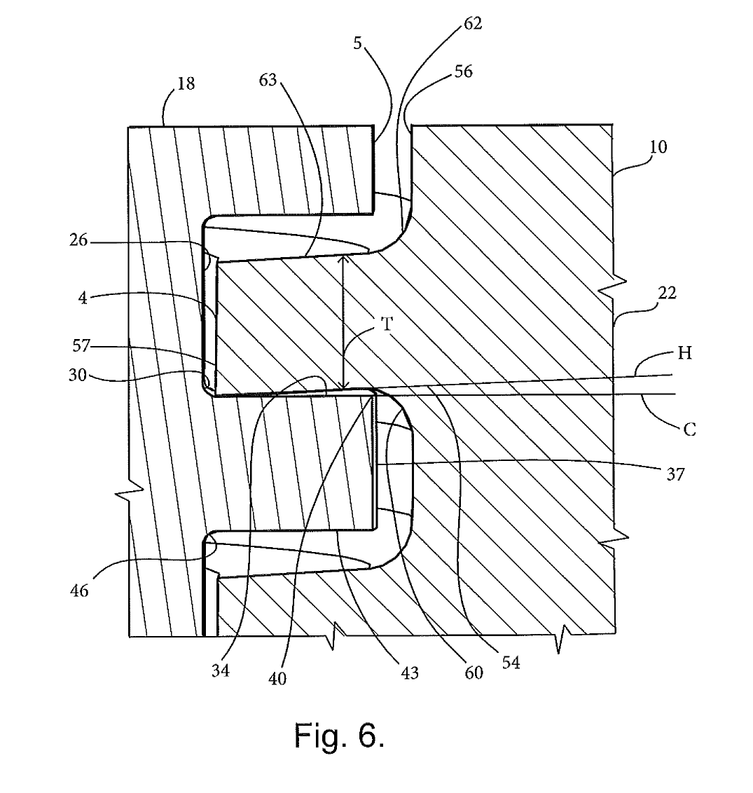

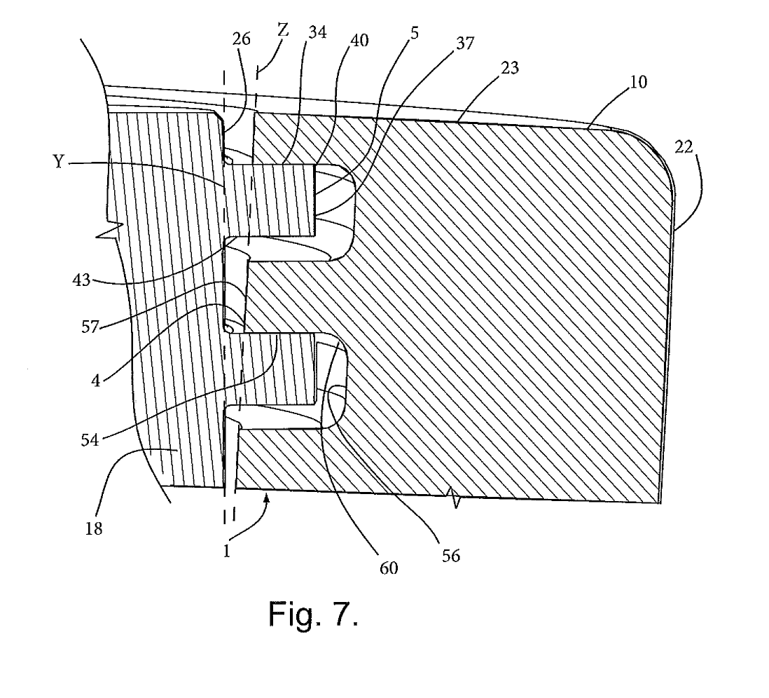

With specific reference to FIGS. 5-7, the thread form 4 of the receiver 10 includes a load flank 54 extending from a root surface 56 toward the receiver axis B and terminating at a crest surface 57. The crest surface 57 is parallel to the root surface 56 and prior to mating with the closure 18, the surfaces 56 and 57 are substantially parallel to the receiver axis B. A radiused corner surface 60 connects the load flank 54 with the root surface 56. At an opposite side of the thread form 4 at root surface 56, another radiused corner surface 62 connects the root surface 56 with a stab flank or surface 63 that also extends to the crest surface 57. Similar to the closure load flank 34 and stab flank 43, the receiver load flank 54 and stab flank 63 are in parallel spaced relation, resulting in the thread form 4 having a uniform thickness measured between the flanks 54 and 63 and perpendicular thereto. With particular reference to FIG. 5, unlike the load flank 34 of the closure 18 that is substantially perpendicular to the closure root surface 26, the receiver load flank 54 is slightly undercut or otherwise disposed at a reverse angle R of less than ninety degrees with an imaginary cylindrical surface defined by the root surface 56. The angle R is chosen to provide an initial contact between the thread form 4 loading flank 54 and the thread form 5 loading flank 34 that is located near the closure root 26 as shown in FIG. 6 and described in greater detail below. With reference to FIG. 6, the angle of the undercut or reverse cut of the flank 54 is indicated with the letter H shown with respect to a horizontal line C extending from the load flank 34 of the closure 18 and perpendicular to the closure axis A. The angles R or H are chosen to accommodate receiver arm pivot (each arm 22) of about one to two degrees, preferably between about 1.2 and about 1.4 degrees for each receiver arm 22 for the loading flanks 34 and 54 to become flush with each other as shown in FIG. 7. Torque applied is generally about 75 to about 90 inch pounds with the illustrated modified square-like threads. Thread pitch can vary, but preferably ranges between about 0.039 inches and about 0.045 inches.

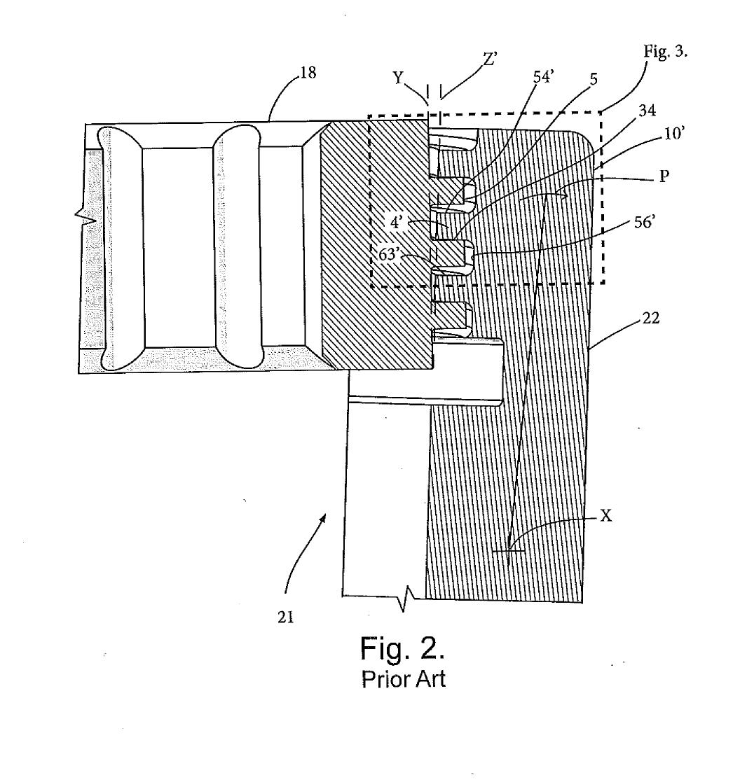

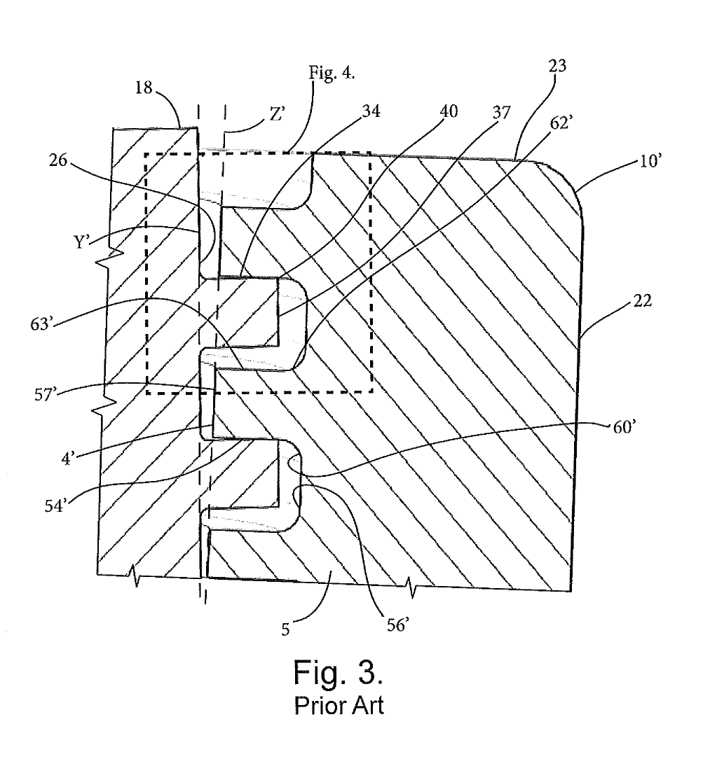

In comparison with the receiver 10′ having a square thread 4′ shown in FIGS. 1-4, the undercut or slightly reverse angled but otherwise substantially square thread-like form 4 counteracts the undesirable outer corner loading of the male square thread 5 of the closure 18 that occurs during mating of the traditional square thread receiver 10′ with the square thread closure 18. As shown in FIGS. 1-4, the receiver 10′ square thread form 4′ includes a load flank 54′, a root surface 56′, a crest surface 57′, opposed radiused corner surfaces 60′ and 62′ and an unloaded stab flank 63′ substantially similar in form and location to the respective load flank 54, root surface 56, crest surface 57, opposed radiused corner surfaces 60 and 62 and stab flank 63 of the thread form 4 previously described herein. However, the square thread form flanks 54′ and 63′ are substantially perpendicular to both the root surface 56′ and the crest surface 57′. In other words, the loading flank 54′ does not include any undercut or reverse angle cut. Although theoretically this should keep all forces axially directed, in open bone anchor receivers utilized with closures having a helical square thread closure, it has been found that a lateral or outward splay of the arms is almost always present and, within reasonable limits, a minor amount of splay is not problematic. In fact, in some embodiments, a small amount of splay may even beneficially reduce torque and desirably improve thrust during early loading of the closure into the receiver. However, Applicant's have found that along with lateral splay, a pivoting or “pivot-splay” is present in square thread embodiments. For example, with reference to FIGS. 2 and 3, the line Y extends substantially vertically along the closure root 26 while the line Z′ extends along the crest 57′. These lines were added to illustrate how the square thread closure 18 is affected by splay of the receiver arms 22 that is not just laterally outward but also resulting in a pivot that changes the stress dynamics on the closure thread loading flank 34 as best shown in FIG. 4. An outward pivot of the receiver arms indicated by the arrow P in FIGS. 2 and 4 occurs as the closure 18 is rotated within the square thread receiver 10′ and prior to tightening. It is believed that a center of rotation X of the splay is located within each receiver upright arm 22 as shown, for example, in FIGS. 2 and 4, and creates unfavorable loading conditions for the closure square thread 5, placing additional stress as well as outward displacement on the closure square thread portion near the corner or edge 50 located near the crest 37 thereof as best shown in FIG. 4.

In contrast, with reference to FIGS. 5-7, although splay still occurs during loading of the closure 18 into the receiver 10 as shown, for example, in FIG. 7, by the lines Y (along the closure root surface 26) and Z (along the receiver crest 57), the thread form 4 according to an embodiment of the invention does not overstress the closure thread form 5. As best shown in FIG. 6, during rotational mating of the closure 18 within the receiver 10 but prior to loading or tightening, the receiver load flank 54 initially engages the closure load flank 34 in an non-parallel relationship, making contact at a location near the closure radiused surface 30 that is adjacent the closure root surface 26. Stated in another way, the discontinuous receiver thread form load flank surface 54 is configured to form an acute angle with the closure load flank surface 34 and engage the closure load flank 34 at a vertex of and acute angle formed by the contact point of the surfaces 34 and 54, such vertex being located substantially medial of the closure splay control ramp. As a practical matter, the initial point of contact between the surfaces 34 and 54 (e.g., the acute angle vertex) should not be located at the root surface 26, but rather at or substantially near where the radiused inner corner surface 30 terminates and transitions into the load flank surface 34 as best shown in FIG. 6. By being more medial than the square thread/square thread contact shown in FIGS. 1-4, the contact location of the surfaces 34 and 54 is more favorably placed on the male thread 5 of the closure 18. Thus, outward pivot-type splay of the receiver arms 22 may occur without unfavorably pre-loading outward portions of the thread 5 of the closure 18 located near the crest 37. As shown, for example, in FIG. 7, the slightly reversed receiver load flank 54 provides for a delay of loading the closure flank 34 outer portion located near the crest 37, but does not impose an upper limit on either splay or outward corner loading. FIG. 7 shows the undercut surface 54 subsequently loaded (tightened) with the load flanks 34 and 54 in substantial full contact or flush with one another. Thus, contact near the closure square thread crest 37 is delayed until late in the loading cycle.

It is foreseen that instead of having the closure loading surface 34 slope forward in cross section from the closure body and the receiver load flank 54 initially extend radially in cross section, as is shown in FIG. 6, the opposite could occur, so that afer final closure both surfaces would be mating and parallel; however, initially the closure loading surface would extend radially in cross section from the closure and the receiver body surface would initially slope rearwardly in cross section from the crest to the root of the receiver thread, preferably the angle rearward required so that the two load surfaces become fully mating and parallel when the closure is fully received in the receiver and fully tightened.

It is to be understood that while certain forms of the present invention have been illustrated and described herein, it is not to be limited to the specific forms or arrangement of parts described and shown.

What is claimed and desired to be secured by Letters Patent is as follows:

1. In a spinal fixation structure having a bone anchor and a closure, the anchor for holding a spinal fixation longitudinal connecting member, the anchor having an open receiver with spaced apart arms defining a longitudinal connecting member receiving channel therebetween and the closure sized for being received within the channel and having a square thread form adapted for rotation and advancement into the channel between the arms to capture a portion of the longitudinal connecting member in the channel, the improvement comprising:
a) a discontinuous receiver guide and advancement structure having a modified square thread form extending helically about and along an inner surface of each receiver arm, the receiver thread form having a load flank with an undercut, the receiver load flank being in initial non-parallel relation with the square thread of the closure and contacting the square thread at a location near a root thereof during initial rotational mating of the closure with the receiver, and wherein upon tightening of the closure with respect to the receiver, the receiver load flank being in substantially full contact with the closure square thread.

2. The improvement of claim 1 wherein the receiver modified square thread includes a stab flank opposed and substantially parallel to the receiver load flank.

3. The improvement of claim 1 wherein the receiver load flank undercut is a reverse angle of up to about four degrees.

4. In a spinal fixation structure having a bone anchor and a closure, the anchor for holding a spinal fixation longitudinal connecting member, the anchor having an open receiver with spaced apart arms defining a longitudinal connecting member receiving channel therebetween and the closure sized for being received within the channel and adapted for rotation and advancement into the channel between the arms to capture a portion of the longitudinal connecting member in the channel, the improvement comprising:
a) a closure guide and advancement structure in the form of a square thread extending helically along the closure and about a central axis of the closure, the form having a first load flank substantially parallel to an opposed first stab flank and a first crest surface disposed substantially perpendicular to both the load flank and the stab flank, the first crest surface running substantially parallel to a root surface of the closure; and
b) a discontinuous receiver guide and advancement structure in the form of a second thread form having a second load flank substantially parallel to an opposed second stab flank and a second crest surface, the second load flank being disposed at an acute angle with respect to the crest surface upon mating of the closure with the receiver, but prior to tightening, a vertex of the acute angle being located substantially near the root of the closure.

5. The improvement of claim 4 wherein the acute angle configured in response to a degree of outward pivot-splay by the receiver during mating with the closure.
